(19) United States
(12) Patent Application Publication
(10) Pub. No.: US 2023/0087299 A1
(43) Pub. Date: Mar. 23, 2023

(54) SYSTEM AND METHOD FOR ENDO-PHENOTYPING AND RISK STRATFYING OBSTRUCTIVE SLEEP APNEA

(71) Applicant: The Brigham and Women's Hospital, Inc., Boston, MA (US)

(72) Inventors: David Andrew WELLMAN, Boston, MA (US); Scott Aaron SANDS, Boston, MA (US); Ali AZARBARZIN, Boston, MA (US)

(21) Appl. No.: 17/922,661

(22) PCT Filed: May 3, 2021
(86) PCT No.: PCT/US2021/030473

§ 371 Date: Nov. 1, 2022

Related U.S. Application Data
(60) Provisional application No. 63/019,066, filed on May 1, 2020.

(51) Int. Cl. G16H 20/40; G16H 50/20

(52) U.S. Cl. CPC G16H 20/40; G16H 50/20

(57) ABSTRACT

A method for endo-phenotyping and risk stratifying obstructive sleep apnea (OSA) incudes acquiring signals associated with breathing over a period of time from a subject. In some embodiments, the signals associated with breathing are oxygen saturation signals (SpO₂) that can be acquired using a SpO₂ sensor. In some embodiments, the signals associated with breathing are snoring signals that can be acquired using an acoustic sensor. The method further includes determining at least one endo-phenotype of OSA using the acquired signals associated with breathing. In some embodiments, SpO₂ signals can be used to determine endo-phenotypes including pharyngeal collapsibility, arousal threshold, ventilatory instability, hypoxic burden, and heart rate burden. In some embodiments, snoring signals can be used to determine endo-phenotypes including a site of airway collapse.

### CROSS-REFERENCE TO RELATED APPLICATIONS

This application is based on, claims priority to, and incorporates herein by reference in its entirety U.S. Ser. No. 63/019.066 filed May 1, 2020, and entitled “Wearable Device to Diagnose and Monitor Sleep Apnea.”

### STATEMENT REGARDING FEDERALLY SPONSORED RESEARCH

This technology was made with government support under grant RO1 HL128658 awarded by the National Institutes of Health. The government has certain rights in the technology.

### FIELD

The present disclosure relates generally to systems and methods for diagnosing and monitoring obstructive sleep apnea (OSA) and, more particularly, to a system and method for determining endo-phenotypes and risk stratification for OSA for a subject.

### BACKGROUND

Obstructive sleep apnea (OSA) is a common disorder characterized by recurrent upper airway collapse (e.g., partial or complete obstructions) during sleep that leads to sleep fragmentation and sympathetic activation. Obstructive Sleep Apnea (OSA) is a common and debilitating disorder. The partial or complete obstructions of the upper airway may be due to collapse of one or more pharyngeal structures (velum, oropharyngeal lateral walls (OPLW), tongue base, and/or epiglottis). Untreated OSA has been associated with neurocognitive problems (daytime sleepiness, reduced attention), cardiovascular disease (CVD), heart failure, and metabolic complications (hypertension, diabetes, strokes).

The field of sleep medicine typically relies on quantifying the frequency of partial (hypopnea) and complete (apnea) airway obstruction observed during sleep (e.g., apnea-hypopnea index, AHI) to diagnose OSA and quantify its severity. However, AHI has been found to only modestly predict mortality or may provide inconsistent results regarding mortality with regard to certain health outcome such as heart failure. One interpretation is that the AHI does not accurately capture the disease burden presented by OSA and, as such, does not reflect the factors that adversely affect the cardiovascular system. AHI provides a simple count of the number of obstructive episodes per hour of sleep, without regard for the duration and the depth of the ventilatory disturbance or blood gas changes. Other metrics that have been used to characterize severity of OSA are measures of nocturnal hypoxemia, such as the percentage of time during sleep with an oxygen saturation below 90% (TST90), hove more strongly predicted CVD. However, the TST90 and similar metrics characterize not only the intermittent hypoxemia occurring secondary to obstructive events but also persistent hypoxemia, such as from chronic obstructive pulmonary disease (COPD), which are unrelated to upper airway obstruction and OSA. Therefore, the characterization of OSA severity using frequency-based metrics (e.g., AHI) or nonspecific metrics (e.g., percent time with oxyhemoglobin saturation [SₚO₂]<90%) may be inadequate.

Continuous positive airway pressure (CPAP) is an effective treatment for OSA but suffers from poor adherence rates. Alternatives to CPAP are available, such as mandibular advancement devices (MAD), hypoglossal nerve stimulation (HGNS), pharyngeal surgery, and positional therapy. However, these treatments work better on some sites of collapse than others, i.e., they are site-specific. For example, MADs seem to work better for tongue and epiglottic collapse than oropharyngeal lateral walls (OPLW) collapse or complete concentric obstruction of the velum. Thus, knowing the site of collapse could improve patient selection for these alternative treatments. The problem, though, is that determination of the site of collapse is typically both costly and invasive.

The usual method for determining the site of pharyngeal collapse in OSA is drug-induced sleep endoscopy (DISE). DISE provides critical anatomical information for surgeons. However, it has several disadvantages. First, it is expensive because it must be performed in the operating room with an otolaryngologist and an anesthesiologist. Second, it is invasive and requires inserting an endoscope through the nose and into the pharynx. Third, DISE is not the natural sleeping state. Rather, sleep is induced with propofol or midazolam, and it only lasts a few minutes. As a result, DISE is rarely performed in the typical workup of OSA, and treatment decisions are seldom made with knowledge of the site of obstruction. If CPAP is tolerated, then this is not a problem. However, if alternative treatments are considered, then the site of obstruction could be valuable.

It would be desirable to provide a system and method for endo-phenotyping and risk stratification of OSA that utilizes oxygen saturation signals and snoring signals. The endo-phenotypes may be used to quantify the severity of OSA in a subject and determine a treatment for OSA for the subject.

### SUMMARY

In accordance with an embodiment, a method for endo-phenotyping obstructive sleep apnea (OSA) for a subject includes acquiring oxygen saturation (SpO₂) signals over a period of time from a subject with a SpO₂ sensor, ensemble averaging the SpO₂ signals to remove noise using a processor, and determining at least one endo-phenotype for OSA based on the ensemble averaged SpO₂ signals. The at least one endo-phenotype of OSA can be one or more of pharyngeal collapsibility, arousal threshold, and ventilatory instability. The method further includes determining a treatment for the subject based on the at least one endo-phenotype for OSA.

In accordance with another embodiment, a method for determining a site of airway collapse for obstructive sleep apnea (OSA) in a subject includes acquiring snoring signals from a subject over a period of time using an acoustic sensor. The snoring signals can be associated with a plurality of snores. The method further includes generating a power spectral density (PSD) plot for each snore in the plurality of snores, determining, using the processor, at least one snoring feature for each snore in the plurality of snores based on the snoring signals and the PSD plot, providing the at least one snoring feature for each snore to a machine learning model, and determining a site of airway collapse for each snore in the plurality of snores using the machine learning model.

### BRIEF DESCRIPTION OF THE DRAWINGS

The present disclosure will hereafter be described with reference to the accompanying drawings, wherein like reference numerals denote like elements.

FIG.1 is a block diagram of a system for endo-phenotyping and risk stratifying obstructive sleep apnea (OSA) in accordance with an embodiment;

FIG. 2 illustrates a method for endo-phenotyping and risk stratifying obstructive sleep apnea (OSA) in accordance with an embodiment;

FIG. 3 illustrates an example simulation of breathing for a subject including a ventilation trace and corresponding SpO₂ trace in accordance with an embodiment;

FIG. 4 illustrates a method for determining endo-phenotypes for OSA of a subject based on SpO₂ signals in accordance with an embodiment;

FIG. 5 illustrates two example SpO₂ curves showing the determination of pharyngeal collapsibility in accordance with an embodiment;

FIG. 6 illustrates two example SpO₂ curves showing the determination of arousal threshold in accordance with an embodiment;

FIG. 7 illustrates an example SpO₂ curve showing the determination of ventilatory instability (loop gain) in accordance with an embodiment; and

FIGS. 8A-8C illustrate an example determination of hypoxic burden in accordance with an embodiment;

FIG. 9 shows example spectrograms of the different sounds that can be produced by different sites (or structures) in the pharyngeal airway in accordance with an embodiment;

FIG. 10 illustrates a method for determining a site of airway collapse based on snoring signals in accordance with an embodiment; and

FIG. 11 is a block diagram of an example computer system in accordance with an embodiment.

### DETAILED DESCRIPTION

The present disclosure describes systems and methods for endo-phenotyping and risk stratification of obstructive sleep apnea (OSA) for a subject based on oxygen saturation (SpO₂) signals and snoring signals from the subject. In some embodiments, the SpO₂ signals are analyzed to determine endotypes of OSA such as, for example, pharyngeal collapsibility, arousal threshold, ventilatory control sensitivity (loop gain) and phenotypes of OSA such as hypoxic burden, and heart rate burden or heart rate response (ΔHR). In some embodiments, the identified endotypes and phenotypes may be used to determine a treatment for the OSA of the subject (e.g., continuous positive airway pressure (CPAP), oral appliance therapy, upper airway surgery, positional therapy, pharmacotherapy). Advantageously, SpO₂ is easy to monitor (e.g., using a pulse oximeter), and may be more accurate than airflow measurements. Airflow measurements may be acquired sing a nasal cannula. However, nasal cannulas cannot assess oral breathing, which is common in OSA. The nasal cannula signal is unstable because the cannula frequently moves inside the nose or becomes dislodged altogether, which can lead to surreptitious changes in airflow. The airflow signals are uncalibrated which can make it hard to know what the baseline value is. In contrast, SpO₂ signals are completely resistant to the effects of oral breathing and are stable signals. SpO₂ signals are calibrated and it is easy to determine a baseline awake value, which can allow for more precise determination of changes due to upper airway obstruction. In addition, desaturation is the likely mechanism through which partial (hypopnea) and complete (apnea) airway obstructions produce cardiovascular disease and other harmful consequences (e.g., sleepiness, neurocognitive deficits, metabolic abnormalities).

In some embodiments, the hypoxic burden and the heart rate burden or heart rate response (ΔHR) may be used risk stratify the OSA for the subject, for example, to determine the risk of mortality from cardiovascular disease or heart failure. In contrast to the apnea-hypopnea index, the hypoxic burden and ΔHR metrics are advantageously based on the SpO₂ signals which take into account not only the frequency of apneas (events) but also the length of the events (10 seconds vs. 30 seconds) and the depth of the events (mild airway closure vs complete airway closure).

In some embodiments, snoring signals (acoustic signals/sound) may advantageously be analyzed to identify the site or site(s) of airway collapse for OSA in a subject. For example, the pharyngeal airway can collapse at one or more of the following sites: the velum (or soft palate), the oropharyngeal lateral walls (OPLW), the tongue base, and the epiglottis. Each of these sites has different mechanical properties that can produce different sounds (e.g. clicks, snorts, chirps) or snoring frequencies when collapsing. In some embodiments, a machine learning model may be configured to predict a site of an airway collapse based on snoring features identified from the snoring signals. In some embodiments, the identified site(s) of collapse may be used to determine a treatment for the OSA of the subject (e.g., mandibular advancement devices (MAD), hypoglossal nerve stimulation (HGNS), pharyngeal surgery, and positional therapy).

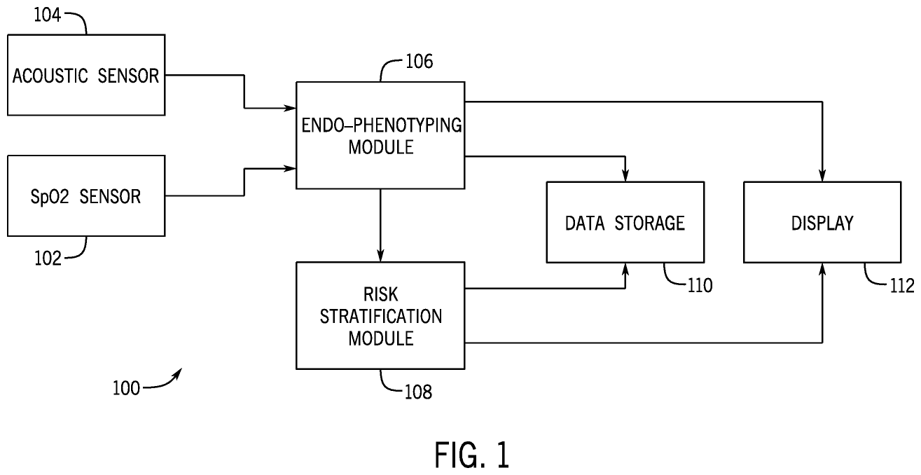

FIG. 1 is a block diagram of a system for endo-phenotyping and risk stratifying obstructive sleep apnea (OSA) in accordance with an embodiment. In FIG. 1, the system 100 includes a SpO₂ sensor 102, an acoustic sensor 104, an endo-phenotyping module 106, a risk stratification module 108, data storage 110 and a display 112. In various embodiments, elements of system 100 may be implemented in the same device. In other embodiments, various elements are implemented in different locations or devices and may be in signal communication via wired or wireless connections. For example, the SpO₂ sensor 102 may be part of a pulse oximetry system, the acoustic sensor 104 may be a microphone positioned near the subject, and the endo-phenotyping module 106, risk stratification module 108, may be implemented on a processor (e.g., on a device for monitoring breathing or a computer system). In some embodiments, various elements of system 100 may be implemented in a wearable device such as a watch, a patch, a vest, etc.).

As mentioned, the endo-phenotyping module 106 and risk stratification module 108, may be implemented on a processor. In some implementations, the processor may be included in any general-purpose computing system or device, such as a personal computer, workstation, cellular phone, smartphone, laptop, tablet, or the like. The processor may include any suitable hardware and components designed or capable of carrying out a variety of processing and control tasks, including steps for endo-phenotyping and risk stratification of obstructive sleep apnea for a subject based on oxygen saturation (SpO₂) signals and snoring signals from the subject. For example, the processor may include a programmable processor or combination of programmable processors, such as central processing units (CPUs), graphics processing units (GPUs), and the like. In some implementations, the processor may be configured to execute instructions stored in a non-transitory computer readable-media. In this regard, the processor may be any device or system designed to integrate a variety of software, hardware, capabilities and functionalities. Alternatively, and by way of particular configurations and programming, the processor may be a special-purpose system or device. For instance, such special-purpose system or device may include one or more dedicated processing units or modules that may be configured (e.g., hardwired, or pre-programmed) to carry out steps, in accordance with aspects of the present disclosure.

The SpO₂ sensor 102 is configured to measure oxygen saturation (SpO₂ signals) of a subject over a period pf time during sleep. The SpO₂ sensor 102 provides the SpO₂ signals to the endo-phenotyping module 106. In an embodiment, the SpO₂ sensor may be implemented in a pulse oximetry device and the pulse oximetry device may also be used to acquire a pulse rate signal that may be provided to the endo-phenotyping module 106. The acoustic sensor 104 is configured to detect and measure snoring sounds from the subject over a period of time during sleep. The acoustic sensor may be for example, a microphone positioned in proximity to the subject. The acoustic sensor provides the detected snoring signals to the endo-phenotyping module 106. In some embodiments, the SpO₂ signals and the snoring signals are measured during the same period of time. In some embodiments, the SpO₂ signals and snoring signal are acquired during different periods of time. In an embodiment the endo-phenotyping module 106 may retrieve the SpO₂, pulse rate, and acoustic signals from data storage, for example, data storage 110 or data storage in another computer system. The endo-phenotyping module 106 may be configured to determine one or more endotypes and phenotypes of OSA based on at least one of the SpO₂ signals acquired by the SpO₂ sensor 102 and the snoring signals acquired by the acoustic sensor 104 as discussed further below. In an embodiment, the endo-phenotyping module 106 may also be configured to determine one or more endotypes or phenotypes f KSA based on a pulse rate signal. In some embodiments, the endotypes and phenotypes of OSA can include, for example, pharyngeal collapsibility, arousal threshold, ventilatory instability (loop gain), hypoxic burden, heart rate burden or heart rate respnse (ΔHR), and site of airway collapse. In an embodiment, one or more of the hypoxic burden and the heart rate burden (ΔHR) metrics may be provided to the risk stratification module 108. The risk stratification module 108 may be configured to determine the severity of the OSA and predict the risk of certain health outcomes for the subject such as, for example, cardiovascular disease and heart failure. The determined endotypes, phenotypes, and risk stratification may be stored in data storage 110 coupled to the endo-phenotype module 106 and the risk stratification module 108. In addition, the endotypes, phenotypes, and risk stratification may be displayed on a display 112 coupled to the endo-phenotype module 106 and the risk stratification module 108.

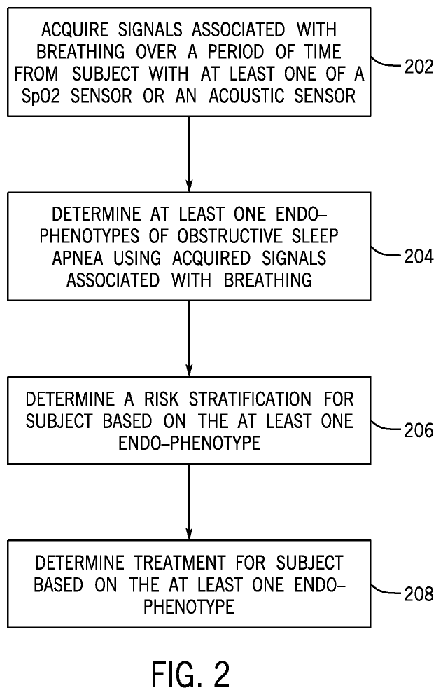

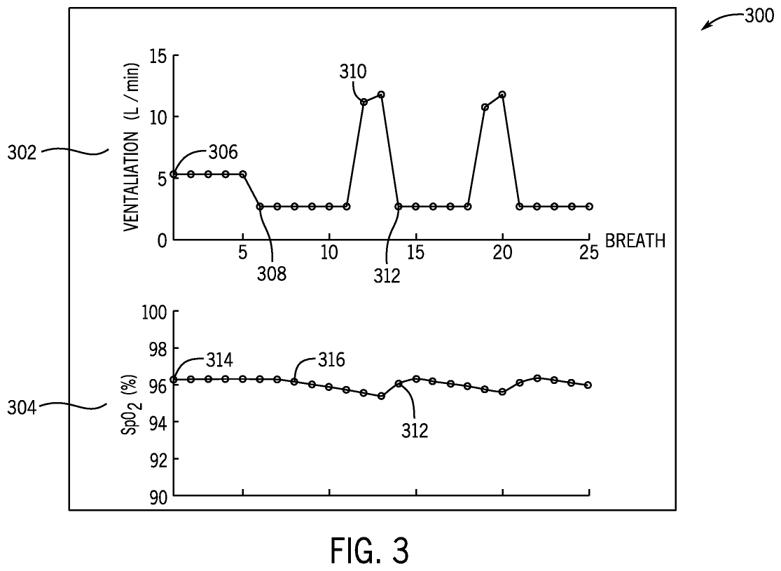

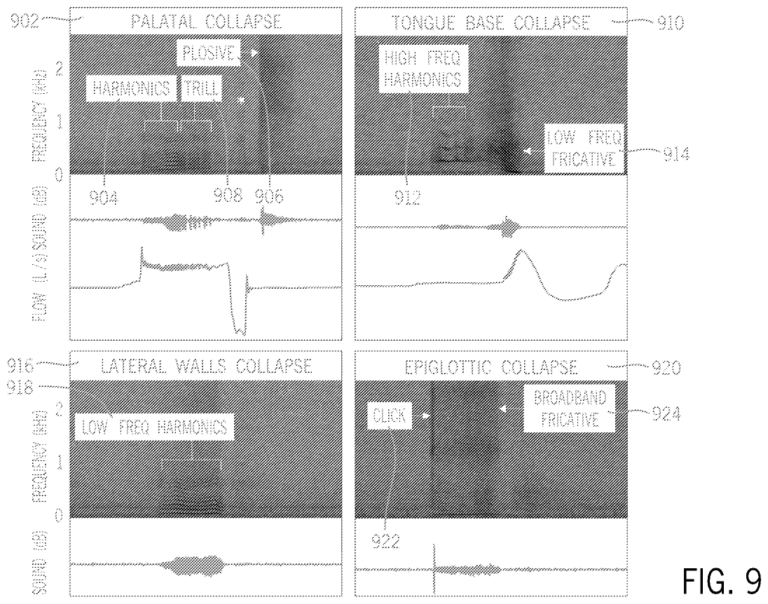

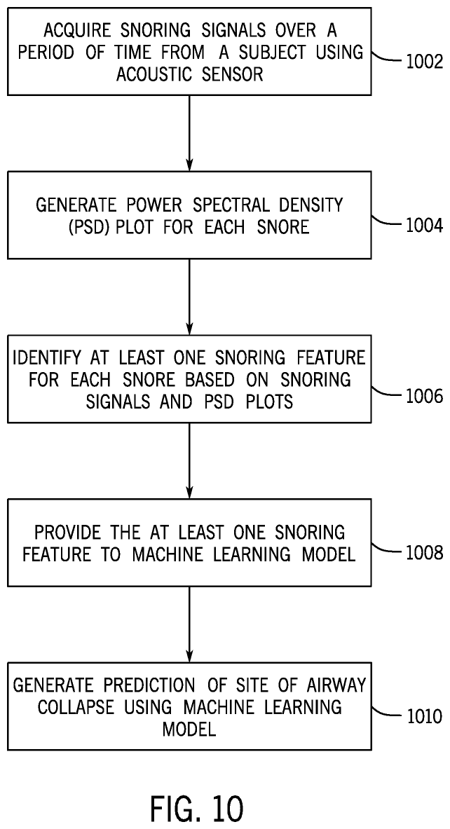

As mentioned above, the endo-phenotyping module 106 may be configured to determine one or more endotypes and phenotypes of OSA based on at least one of the SpO₂ signals and the snoring signals, and the risk stratification module 108 may be configured to determine the severity of the OSA and predict the risk of certain health outcomes for the subject. FIG. 2 illustrates a method for endo-phenotyping and risk stratifying obstructive sleep apnea (OSA) in accordance with an embodiment. At block 202, signals associated with breathing of a subject are acquired from the subject over a period of time using at least one of a SpO₂ sensor (e.g., SpO₂ sensor 102 shown in FIG. 1) and an acoustic sensor (e.g., acoustic sensor 104 (shown in FIG. 1) In another embodiment, the signals associated with breathing may be retrieved from data storage, for example, data storage 110 shown in FIG. 1. The acquired signals associated with breathing can include, for example, oxygen saturation (SpO₂) signals and snoring signals (acoustic signals/sound). In an embodiment, the SpO₂ sensor may be implemented in a pulse oximetry device and the pulse oximetry device may also be used to acquire a pulse rate signal. The signals associated with breathing may be acquired over a period of time during sleep of the subject. At block 204, at least one endo-phenotype of OSA is determined using the acquired signals associated with breathing. In some embodiments, the signals acquired at block 202 include SpO₂ signals that may be used to determine one or more of pharyngeal collapsibility, arousal threshold, ventilatory instability (loop gain), hypoxic burden, and heart rate burden (ΔHR) as described further below with respect to FIGS. 3-8. In some embodiments, the signals acquired at block 202 include snoring signals that may be used to determine a site of airway collapse in OSA for the subject as described further below with respect to FIG. 9-10.

At block 208, a risk stratification for OSA for the subject may be determined based on at least one of the determined endo-phenotypes such as, for example, hypoxic burden or heart rate burden (or heart rate response). In an embodiment, the hypoxic burden and/or the heart rate burden may be used to predict the risk of certain health outcomes for the subject such as, for example, cardiovascular disease and heart failure. At block 210, a treatment for OSA of the subject may be determined or informed based on at least one of the determined endo-phenotypes such as, for example, pharyngeal collapsibility, arousal threshold, ventilatory instability (loop gain), and site of airway collapse. Examples, of various types of treatments for OSA include, but are not limited to, continuous positive airway pressure (CPAP), oral appliance therapy, upper airway surgery, positional therapy, and pharmacotherapy.

CPAP mechanically splints the airway open with positive pressure applied through a tight fitting face mask. CPC works for most patients, however it may be difficult to tolerate for many patients. In an embodiment, CPAP may be the only effective treatment for OSA patients who have a highly collapsible airway. Accordingly, is may be advantageous to determine the pharyngeal collapsibility to identify subjects with a highly collapsible airway for CPAP treatment and avoid unnecessary application of alternative treatments that are unlikely to be effective. Oral appliances pull the mandible (lower jaw) anteriorly, thereby moving the upper airway structures (tongue mostly) anteriorly and opening the pharyngeal airspace. Many studies have indicated that oral appliances may work best for patients with tongue base collapse, a low loop gain, and mild pharyngeal collapsibility. Upper airway surgery can be divided into three categories: hard tissue surgeries, soft tissue surgeries, and hypoglossal nerve stimulation. Hard tissue surgeries, such as maxillomandibular advancement, may be effective but are rarely performed because they are invasive. Soft tissue surgeries target a specific structure, such as the palate, uvula, tongue and/or epiglottis. Therefore, they are inherently site specific and identifying the site of collapse may improve outcomes. Accordingly, a site of airway collapse may be identified from the snoring signals, as discussed further below, and a subsequent surgery performed on the identified site of airway collapse. Advantageously, using the snoring signals to identify the site of airway collapse is not invasive as is the case with existing methods that involve endoscopy. Hypoglossal nerve stimulation (HGNS) is contraindicated in patients with complete concentric collapse of the palate, which may be identified using the snoring signals. HGNS has been shown to work better for tongue collapse. In some individuals, OSA only occurs when sleeping in the supine position. Studies have indicated that positional OSA may often be caused by collapse of the epiglottis. Accordingly, determination of the site of the airway collapse as the epiglottis can be used to inform the use of positional therapy for the subject. Mandibular advancement devices (MADs) have been shown to work better for tongue and epiglottal collapse. In pharmacotherapy, drugs used for therapy may be designed to work on a particular endotype of OSA. For example, drugs to activate the pharyngeal muscles during sleep may work best in patients with a mildly collapsible airway, drugs for reducing loop gain may work best in patients with a high loop gain, and sedatives/hypnotics may work best in patients with a low arousal threshold.

As mentioned above, in some embodiments, SpO₂ signals may be used to determine one or more endotypes and phenotypes including pharyngeal collapsibility, arousal threshold, ventilatory instability (loop gain), hypoxic burden, and heart rate burden (ΔHR). Advantageously, SpO₂ can be used to monitor ventilation. Changes in ventilation directly produce change in SpO₂ as illustrated in FIG. 3. FIG. 3 illustrates an example simulation of breathing 300 for a subject including a ventilation trace 302 and corresponding SpO₂ trace 304 in accordance with an embodiment. In the example ventilation graph 302, the ventilation is initially at a resting, or eupneic, level 306 of approximately 5.1 L/min. On breath six 308 in this example, the subject falls asleep and the airway obstructs partially, leading to a reduction in ventilation to approximately 2.6 L/min. On breath twelve 310 in this example, the subject arouses from sleep, the airway opens and ventilation overshoots to approximately 12 L/min. Sleep resumes on the fourteenth breath 312, and the airway again obstructs, and so on. Therefore, this example is a simulation of obstructive hypopneas. The associated SₚO₂ is shown in the lower graph 304. The resting (eupneic) SpO₂ 314 is approximately 96.3%. On breath eight 316, the SpO₂ begins to decrease due to the reduction in ventilation. In this example, there is a two breath lag between the change in ventilation and the change in SₚO₂ because it takes time for the deoxygenated blood to reach, for example, the finger. In this example, while there is an abrupt (square wave) drop in ventilation, there is a ramp-like reduction in SₚO₂. The increase in ventilation on breath twelve 310 leads to an increase in SₚO₂ on breath fourteen 312, again because of the two-breath delay. Accordingly, changes in ventilation directly produce change in SpO₂ as illustrated and SpO₂ can be used to monitor ventilation.

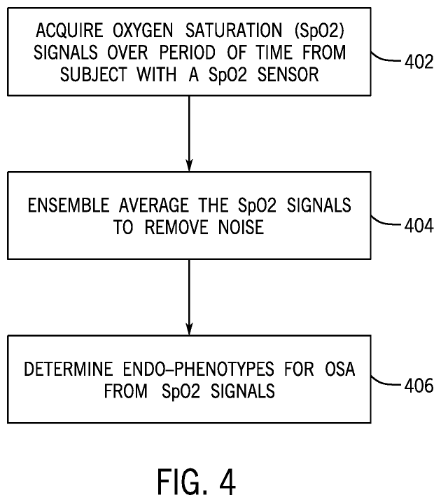

FIG. 4 illustrates a method for determining endo-phenotypes for OSA of a subject based on SpO₂ signals in accordance with an embodiment. At block 402, oxygen saturation (SpO₂) signals are acquired over a period of time from a subject using a SpO₂ sensor (e.g., SpO₂ sensor 102 shown in FIG. 1). The SpO₂ signals may be acquired over a period of time during sleep of the subject. At block 404, the SpO₂ signals are ensemble averaged to remove noise. Typically, the sensors or hardware used to detect SpO₂ signals may designed to detect oxygen changes that occur slowly over several minutes. Consequently, the SpO₂ signals may be heavily filtered, which can chop off the transient changers that occur over the 10-20 seconds due to an apnea or hypopnea (i.e., a respiratory event). This can present problems when determining parameters such as, for example, nadir oxygen saturation due to transient apneas. Ensemble averaging provides a method for smoothing the SpO₂ signal without using filters. The ensemble averaging method also does not obscure nadir oxygen saturation transients. In the first step to ensemble average the SpO₂ signal, the terminal time point (associated with the terminal breath) in each hypopnea and apnea is identified. The terminal time point (or breath) is the last time point (or breath) of the respiratory event (i.e., hypopnea or apnea). Second, the signal for each respiratory events (e.g., hypopnea or apnea) are spliced out of the SpO₂ signal and stacked on top of one another, lining them up by their terminal time points. Finally, the signals for each respiratory event are ensemble averaged to produce an “average” respiratory event with the noise removed.

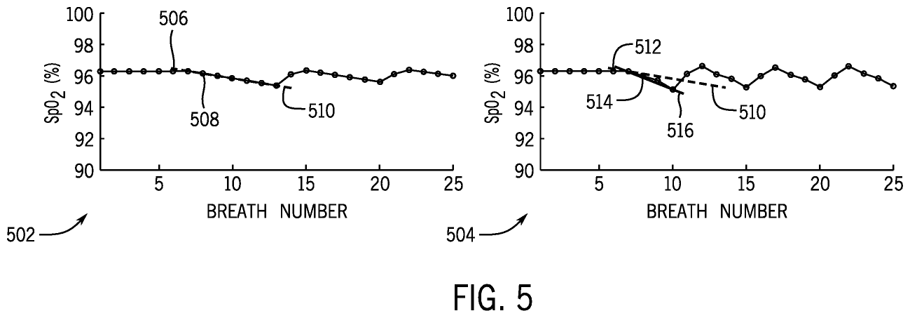

Once the ensemble averaged SpO₂ signal is generated at block 404, at least one endo-phenotype for OSA may be determined at block 406 based on the ensemble averaged SpO₂ signal. As mentioned above, the at least one endotype or phenotype may include, for example, the pharyngeal collapsibility, arousal threshold, ventilatory instability (loop gain), hypoxic burden, and heart rate burden (ΔHR). The pharyngeal collapsibility may be determined or estimated using the slope of the oxygen desaturation curve. Greater collapsibility may be manifest by a faster slope of desaturation (desaturation slope). Typically, the greater the loss of airflow during a respiratory event (e.g., hypopnea or apnea), the faster the fall in SpO₂. FIG. 5 illustrates two example SpO₂ curves showing the determination of pharyngeal collapsibility in accordance with an embodiment. In curve 502, upon sleep onset at breath (or time point) six 506, the pharynx obstructs partially (hypopnea) and reduces ventilation (not shown) for six breaths. In response to the reduction in ventilation, the SpO₂ signal begins to decrease at breath (or time point) eight 508 and continues to decrease for six breaths. The slope 510 of the oxygen desaturation curve (reduction in the SpO₂ signal) can be used as a marker of how collapsible the airway is and provides an estimate of the collapsibility. In an embodiment, the slope of the oxygen desaturation curve may be calculated using the 10-90th centile method. A steeper drop (or slope) in the SpO₂ signal can represent a worse (or more collapsed) airway. For example, in curve 504, upon sleep onset at breath (or time point) six 512, the pharynx obstructs completely (apnea) and reduces ventilation (not shown) for six breaths. The complete obstruction will cause a larger reduction in ventilation than the partial obstruction. In response to the reduction in ventilation, the SpO₂ signal begins to decrease at breath (or time point) eight 514 and continues to decrease for six breaths. The slope 516 of the oxygen desaturation curve (reduction in the SpO₂ signal) for the complete obstruction is steeper than the slope of the desaturation curve 510 for the partial obstruction of curve 502. Accordingly, greater desaturation can be a surrogate of greater collapsibility. In an embodiment, the depth of the respiratory event (hypopnea or apnea) also correlates with the oxygen desaturation slope.

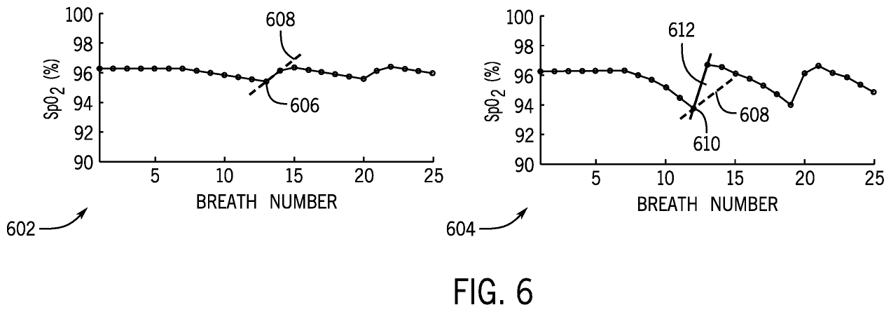

The arousal threshold may be determined or estimated using the upslope of the ensemble averaged SpO₂ signal after an arousal and before the next respiratory event. FIG. 6 illustrates two example SpO₂ curves showing the determination of arousal threshold in accordance with an embodiment. Curve 602 illustrates SpO₂ for respiratory event for a first subject. At breath (or time point) thirteen 606 on curve 602, the SpO₂ signal begins to increase after the subject has aroused from a respiratory event. A subject may arouse because the ventilatory drive has exceeded the arousal threshold. Ventilatory drive may be defined as the amount of ventilation the patient wants. Sometimes the ventilation is equal to the ventilatory drive, such as when the airway is patent and other times, such as during airway obstruction, the ventilation is less than ventilatory drive. During an airway obstruction (breaths eight to thirteen), the patient cannot achieve the desired ventilation. Consequently, carbon dioxide builds up, SpO₂ drops, and the ventilatory drive increases well above the resting ventilation level. Eventually, the ventilatory drive increase to a point that causes the brain to arouse (breath thirteen 606), and open the airway. When the airway opens, ventilation can once again meet the ventilatory drive demand. The ventilation upon airway opening may be a surrogate measure of the arousal threshold and the start of the upslope 608 of the SpO₂ curve after arousal (at breath thirteen 606), which may be a surrogate for the ventilation when the airway opens, may also be used as a measure of the arousal threshold. A higher arousal threshold may promote a longer time for rising ventilatory drive to trigger arousal from sleep, and subsequent respiratory event termination, and thus may lead to a greater event related loss of SpO₂. Accordingly, a greater respiratory event duration (or desaturation duration) may be associated with a higher arousal threshold. Curve 604 illustrates SpO₂ for a respiratory event for a second subject. The second subject has a much higher arousal threshold than the first subject as illustrated by the steeper slope 612 of the SpO₂ signal after an arousal at the arousal threshold indicated by breath (or time point) twelve 610. In some embodiments, pulse rate may also be used to provide additional insight into the arousal threshold. Greater increases in pulse rate to respiratory (desaturation) events may reflect the presence rather than absence of arousals from sleep. In an embodiment, pulse rate surges (e.g. 6 bpm) following desaturation may be used to indicate arousals and estimate a surrogate measure of the arousal threshold (median SpO₂ prior to arousal).

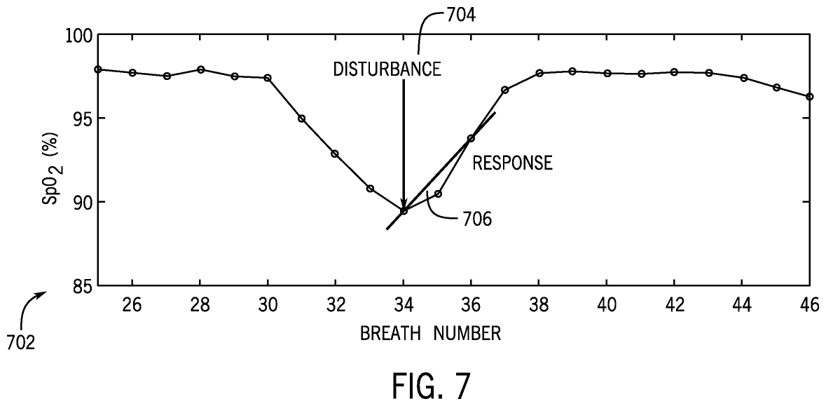

The ventilatory instability (loop gain) or ventilatory control sensitivity may also be determined or estimated from the ensemble averaged SpO₂ signal. Loop gain is the gain (or sensitivity) of the ventilatory control feedback loop. In one embodiment, the loop gain may be defined as the ventilatory drive response to a ventilatory disturbance. The disturbance is the cumulative reduction in ventilation during the hypopnea/apnea and the ventilatory response is the ventilatory overshoot after the airway reopens. In terms of the SpO₂ signal, these parameters correspond to the drop in SpO₂ below baseline (the disturbance) and the slope of the oxygen recovery. FIG. 7 illustrates an example SpO₂ curve showing the determination of ventilatory instability (loop gain) in accordance with an embodiment. In FIG. 7, the disturbance is represented by the drop in ensemble averaged SpO₂ signal (curve 702) below baseline at breath (or time point) 704. The response is shown by the slope 706 of the oxygen recovery after airway reopening and may be used to reflect ventilatory instability. For example, the SpO₂ signal across the time course of events is a manifestation of the loss and recovery of airflow. Loop gain (instability) may be estimated as the magnitude of the airflow during the recovery phase divided by the magnitude of the event-related loss of airflow. Thus, elevated loop gain may be observed in the SpO₂ signal using the speed (slope 706) of the post-event recovery versus the depth (nadir SpO₂) of desaturation.

In another embodiment, consistency of the desaturation timing may reflect increased ventilatory instability. A higher ventilatory instability may yield more periodic respiratory events and attendant desaturation events. A reduced coefficient of variation of event-to-event timing E_{cov} is a predictor of elevated instability (loop gain), and central versus obstructive sleep apnea in patients with heart failure. Specifically, higher loop gain may be associated with a lower E_{cov}. The presence of central versus obstructive sleep apnea may also associated with lower E_{cov}. In some embodiments, an alternative version of E_{cov} may be derived from oximetry alone and may similarly reveal increased ventilatory instability. In this embodiment, E_{cov} for desaturation may be calculated by first finding the typical event-to-event duration (aligning desaturation events at event termination (i.e., minimal SpO₂). Fourier analysis may be performed in the ensemble averaged SpO₂ signal to find the fundamental cycle period T Next, individual event-to-event durations may be tabulated, and those exceeding a predetermined threshold (e.g., 1.67 T) may be removed. Finally, the mean and standard deviation (SD) event-to-event durations may be calculated to provide E_{cov} (SD/mean). In an embodiment, values of E_{cov} under a predetermined value (e.g., 0.2) may be considered to reflect an elevated ventilatory instability.

In other embodiments, further surrogates of instability using analysis of the variability of the SpO₂ signal that reflect quasi-periodic or narrow bandwidth variability in SpO₂ rather than random broadband variability may be used to reflect ventilatory instability. In some embodiments, snoring signals may also be used to provide additional insight into the ventilatory instability. For example, reduced snoring amplitude may reflect lower ventilatory drive or effort during events. Thus lower snoring sound amplitude (overnight, and prior to desaturation events) may be used to reflect a greater ventilatory instability contribution rather than a collapsibility contribution to sleep apnea.

In some embodiments, the phenotype hypoxic burden may also be determined or estimated from the ensemble averaged SpO₂ signal. Hypoxic burden may be used to, for example, quantify the severity of OSA (e.g., quantify OSA related hypoxemia) and as a predictor of health outcomes (e.g., cardiovascular or heart failure mortality). In an embodiment, hypoxic burden is defined as the area under a desaturation curve of the ensemble averaged SpO₂ signal from a pre-event baseline (e.g., the pre-event SpO₂ may be sued as the baseline). In an embodiment, the hypoxic burden may be categorized (e.g., divided into quintiles) and each category associated with a level of risk of, for example, cardiovascular disease of heart failure, for a subject. Advantageously, the hypoxic burden takes into account the frequency, depth and duration of respiratory events such as sleep related upper airway obstructions (e.g., hypopnea and apnea). Hypoxic burden differs from other metrics that have been used to characterize severity of OSA (e.g., TST90) because it captures transient hypoxemia due to upper airway obstruction, as opposed to sustained reductions in oxygen due to lung disease.

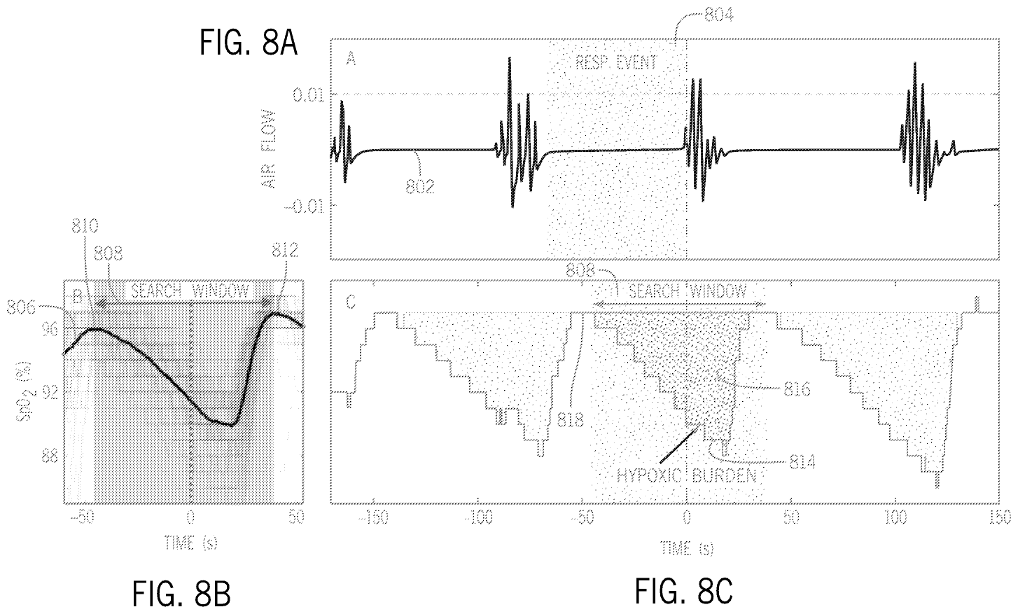

FIGS. 8A-8C illustrate an example determination of hypoxic burden in accordance with an embodiment. The hypoxic burden matric captures the total amount of respiratory event-related hypoxemia over the sleep period. FIG. 8A illustrates an airflow (or ventilation) signal 802 of a subject over a period of time including respiratory events such as the respiratory event define by box 704. For each individually identified respiratory event (e.g., respiratory event 704), such as an apnea or hypopnea, a pre-event baseline saturation is defined as the maximum SpO₂ during the 100s prior to the end of the event. The area under the baseline value may be calculated over a subject-specific search window. FIG. 8B shows an overlaid ensemble averages SpO₂ signal associated with all respiratory events for the subject. The subject-specific search window 808 may be obtained from the ensemble averaged SpO₂ curve 806. As discussed above, the ensemble averaged SpO₂ may be generated for a subject by overlaying the SpO₂ signals for each event with respect to the end of events and averaging the signals. In FIG. 8B, the search window 808 is calculated as the time between two peaks (810, 812) of the ensemble averaged SpO₂ curve 806. The search window 808 may be used to calculate the hypoxic burden for each individual respiratory event for the subject over the sleep time, that is, to calculate the area under the saturation curve within the search window as shown in FIG. 8C. In FIG. 8C, the hypoxic burden 814 (i.e., the area 816 under the desaturation curve from the pre-event baseline 818) for an individual respiratory event within the search window 808 is shown. The total hypoxic burden for the subject may be obtained by summing the individual burdens for each individual respiratory event and then dividing the sum (or total area) by the total sleep time. The units of the hypoxic burden are % minute/hour. For example, a hypoxic burden of 20% min/h is equivalent to 20 minutes of 1% desaturation per hour or 5 minutes of 4% desaturation per hour. In another example, a hypoxic burden of 50% min/h corresponds to a 5% reduction in SpO₂ below baseline for 10 minutes every hour of sleep.

In some embodiments, the phenotype heart rate burden (or heart rate response) (ΔHR) may be determined or estimated from a pulse rate signal. The pulse rate signal may be ensemble averaged in a similar manner as described above with respect to the SpO₂ signal. Heart rate burden (or heart rate response) may be used to, for example, quantify the severity of OSA and as a predictor of health outcomes (e.g., cardiovascular or heart failure mortality). In an embodiment, the heart rate burden may be categorized (e.g., low ΔHR, mid ΔHR, high ΔHR) and each category associated with a level of risk of, for example, cardiovascular disease or heart failure, for a subject. For example, subjects with OSA that demonstrate high (or elevated) heart rate may be at an increased risk for cardiovascular mortality. In some embodiments, the heart rate burden (ΔHR) and the hypoxic burden may be analyzed together to improve risk stratification (e.g., cardiovascular risk stratification) in OSA. The heart rate burden (or heart rate response) is quantified by respiratory event-related changes in pulse rate, ΔHR (or pulse rate response to respiratory events). ΔHR may be defined as the difference between the maximum pulse rate after airway opening (i.e., during a subject-specific search window) and an event-related minimum pulse rate (i.e., minimum pulse rate during respiratory events, e.g., apneas/hypopneas). In some embodiments, the search window may extend from a pre-event minimum to a post-event minimum of the event-related, ensemble averaged pulse rate. The subject-level heart rate burden (ΔHR) may be determined as the mean of all the event-specific ΔHR.

As mentioned above with respect to FIG. 2, in some embodiments the signals associated with breathing acquired at block 202 can include snoring signals that may be used to determine a site of airway collapse in OSA for the subject. In OSA, the pharyngeal airway can collapse at one or more of the following sites: velum (or soft palate), oropharyngeal lateral walls (OPLW), tongue base, or epiglottis. Each of these structures can have different mechanical properties that produce distinct, different sounds (e.g., clicks, snorts, chirps) or snoring frequencies when collapsing (e.g., airway obstruction). FIG. 9 shows example spectrograms of the different sounds that can be produced by different sites (or structures) in the pharyngeal airway in accordance with an embodiment. A spectrogram is a frequency versus time plot, with the power at each frequency represented by shading. The spectrogram 902 illustrates the sounds that may be generated by a velum or palatal collapse. A palatal collapse 902 can produce low frequency harmonics 904 (i.e., sounds waves that resonate at specific frequencies), trills 904 (e.g., sounds like a “rolling r”), and plosives 906 (i.e., abrupt release of pressure, like air let out of a tire). Harmonics occur when a vibrating object, e.g., the velum, vibrates at many different frequencies simultaneously. The harmonic that vibrates at the lowest frequency is called the fundamental frequency. For the velum, the fundamental frequency is about 50 Hz. The plosives 906 can be caused by palatal prolapse, i.e., the palate prolapses into the nasopharynx on expiration. The spectrogram 910 illustrates the sounds that may be generated by a tongue base collapse. A tongue base collapse 910 can exhibit higher frequency harmonics. In addition, there can also be a low frequency fricative 914 (i.e., friction noise produced by turbulent flow through a constriction) during late inspiration, indicating airway opening. A fricative can sound like white noise. The spectrogram 916 illustrates the sounds that may be generated by a collapse of the oropharyngeal lateral walls (OPLW). The OPLW collapse 916 can produce low frequency harmonics 918, but unlike palatal collapse 902, the harmonics tend to be flatter (i.e., the pitch does not vary and are cleaner). The fundamental frequency for OPLW snores is around 100 Hz, giving these snores a higher pitch. The spectrogram illustrates the sounds that may be generated by an epiglottic collapse. The epiglottic collapse 920 can produce a click 922 (associated with (e.g., synchronous with) the flow discontinuity) followed by a broadband fricative sound 924. The clicking sound may correspond to the epiglottis being sucked against the posterior pharyngeal wall. In addition, sometimes there are harmonics following the clicking sound due to air flowing through the aryepiglottic fold.

FIG. 10 illustrates a method for determining a site of airway collapse based on snoring signals in accordance with an embodiment. The method shown in FIG. 10 may be configured to determine the site of collapse using an easily acquired signal (e.g., using a microphone) during natural sleep of a subject. In some embodiments, the method may be implemented in a device that may be used to acquire snoring signal sin the subject's home (rather than an operating room) for the whole night. At block 1002, snoring signals (acoustic signals, snoring sounds) are acquired over a period of time from a subject using an acoustic sensor (e.g., acoustic sensor 104 shown in FIG. 1). The snoring signals may be acquired over a period of time during sleep of the subject and may be associated with a plurality of snores. In an embodiment, the acoustic sensor may be a microphone positioned in proximity to the subject during a period of sleep of the subject. In some embodiments, the snoring signals can represent partial collapses of the airway because snoring can typically only occur when there is airflow. A complete collapse means that the lumen is abolished and there is no airflow. However, it has been shown that almost all subjects with complete collapse exhibit partial collapse leasing into the apnea. Accordingly, in some embodiments, the lead-in breaths of partial collapse may be used to characterize the collapsing structure from snoring sounds.

At block 1004, a power spectral density (PSD) plot is generated for each snore in the snore signals acquired at block 1002. A PSD plot shows the signal amplitude, quantified as the relative log power, of different frequency components. At block 1006, at least one snoring feature is identified for each snore based on the acquired snoring signals and corresponding PSD plots. In some embodiments, the snoring features include, for example, relative power in very low (0-125 Hz), low (250-500 Hz) and high (2-2.5 kHz) frequency bands, formant frequencies, harmonic power, and fundamental frequency. The identified snoring features from the snoring signals and PSD plots may be used to identify or predict a site (or sites) of collapse for the acquired soring signals. The snoring features can differentiate between the different sites of collapse.

At block 1008, the at least one snoring feature for each snore may be provided to a machine learning model. In an embodiment, the machine learning model is a “random forest” machine learning model. The machine learning model may be included in an endo-phenotyping module 106 shown in FIG. 1 and, for example, implemented on a processor as described above with respect to FIG. 1. The machine learning model may be trained using training methods known in the art. In some embodiments, the machine learning model may be trained to identify a plurality of different sites of collapse including, but not limited to velum (or soft palate), OPLW, tongue base, or epiglottis. In an embodiment, the training data may include, for example, a plurality of measured or simulated snoring signals which each include a label for an associated site of airway collapse (e.g., a category) and identified associated snoring features. In some embodiments, the labels include categories that are combinations of two or more sites to incudes categories that address situations where two or more sites that may collapse simultaneously, as discussed further below.

At block 1010, the machine learning model is used to generate a prediction of the site of the airway collapse for each snore based on the snoring features identified from the snoring signals and corresponding PSD plots. As mentioned, the snoring features are provided as input to the machine learning model. Accordingly, the identified snoring features may be used to identify and differentiate between different sites of collapse. In one example, snores resulting from velar and epiglottal collapse can have higher very low frequency power and less low frequency power than snores resulting from OPLW collapse and tongue collapse. Velar collapse may be separated from epiglottic collapse with high frequency power which can be higher in snores resulting from epiglottic collapse. Tongue collapse may be separated from OPLW collapse with high frequency power which can be higher in snores resulting from tongue collapse. In another example, sites of collapse that exhibit power in similar frequency bands may be distinguished because they produce different sounds.

In some embodiments, the machine learning model is configured to identify or determine a plurality of sites of collapse based on the input snoring features. More than one site of collapse may occur in one of two ways. The first way multi-site collapse can occur is that a single structure collapses at a first point in time and then at a later second point in time another, different structure collapses. In this situation, however, one site may predominate across the night and may be used for selecting a treatment. The second way multi-site collapse can occur is that two structure collapse simultaneously. When this happens, it may be possible to discern which one of the multiple collapses is primary, e.g., the tongue pushing the vellum or epiglottis backwards. Studies have shown that most subjects can have a predominant collapsing structure which may be used to resolve issues of multiple collapsing sites. Sometimes, however, multiple (e.g. two) structures may be collapsing simultaneously with neither one being predominant. In an embodiment, a new category may be created to capture the multi-site collapse. For example, if both the velum and the OPLW collapse together, then the collapsing “site” could be labeled, for example, V+O. These “combined sites” can be included in the training data and used for training the machine learning model. As discussed above with respect to FIG. 2, the identified site or sites of collapse may be used to improve selection of treatment for a subject.

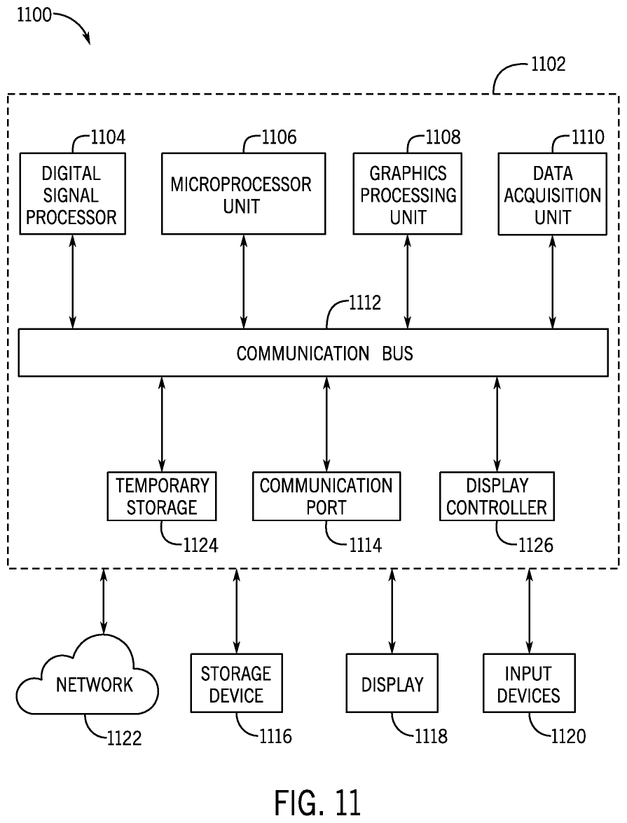

FIG. 11 is a block diagram of an example computer system in accordance with an embodiment. Computer system 1100 may be used to implement the systems and methods described herein. In some embodiments, the computer system 1100 may be a workstation, a notebook computer, a tablet device, a mobile device, a multimedia device, a network server, a mainframe, one or more controllers, one or more microcontrollers, or any other general-purpose or application-specific computing device. The computer system 1100 may operate autonomously or semi-autonomously, or may read executable software instructions from the memory or storage device 1116 or a computer-readable medium (e.g., a hard drive, a CD-ROM, flash memory), or may receive instructions via the input device 1122 from a user, or any other source logically connected to a computer or device, such as another networked computer or server. Thus, in some embodiments, the computer system 1100 can also include any suitable device for reading computer-readable storage media.

Data, such as data acquired with, for example, a SpO₂ sensor or an acoustic sensor may be provided to the computer system 1100 from a data storage device 1116, and these data are received in a processing unit 1102. In some embodiment, the processing unit 1102 includes one or more processors. For example, the processing unit 1102 may include one or more of a digital signal processor (DSP) 1104, a microprocessor unit (MPU) 1106, and a graphics processing unit (GPU) 1108. The processing unit 1102 also includes a data acquisition unit 1110 that is configured to electronically receive data to be processed. The DSP 1104, MPU 1106, GPU 1108, and data acquisition unit 1110 are all coupled to a communication bus 1112. The communication bus 1112 may be, for example, a group of wires, or a hardware used for switching data between the peripherals or between any components in the processing unit 1102. The processing unit 1102 may also include a communication port 1114 in electronic communication with other devices, which may include a storage device 1116, a display 1118, and one or more input devices 1120. Examples of an input device 1120 include, but are not limited to, a keyboard, a mouse, and a touch screen through which a user can provide an input. The storage device 1116 may be configured to store data, which may include data such as, for example, SpO₂ signals, snoring signals, predictions of a site of airway collapse, endo-phenotypes of OSA, risk stratification of OSA, etc., whether these data are provided to, or processed by, the processing unit 1102. The display 1118 may be used to display images and other information, such as, SpO₂ signals, snoring signals, predictions of a site of airway collapse, endo-phenotypes of OSA, risk stratification of OSA patient health data, and so on.

The processing unit 1102 can also be in electronic communication with a network 1122 to transmit and receive data and other information. The communication port 1114 can also be coupled to the processing unit 1102 through a switched central resource, for example the communication bus 1112. The processing unit can also include temporary storage 1124 and a display controller 1126. The temporary storage 1124 is configured to store temporary information. For example, the temporary storage 1124 can be a random access memory.

Computer-executable instructions for endo-phenotyping and risk stratifying obstructive sleep apnea (OSA) according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. A method for endo-phenotyping obstructive sleep apnea (OSA) for a subject, the method comprising:
acquiring oxygen saturation (SpO₂) signals over a period of time from a subject with a SpO₂ sensor;
ensemble averaging the SpO₂ signals to remove noise using a processor;
determining, using the processor, at least one endo-phenotype for OSA based on the ensemble averaged SpO₂ signals, wherein the at least one endo-phenotype of OSA is one or more of pharyngeal collapsibility, arousal threshold, and ventilatory instability; and
determining a treatment for the subject based on the at least one endo-phenotype for OSA.

2. The method according to claim 1, wherein the at least one endo-phenotype is one or more of hypoxic burden and heart rate burden (ΔHR).

3. The method according to claim 2, further comprising determining a risk stratification of OSA based on at least one of the hypoxic burden and the heart rate burden (ΔHR).

4. The method according to claim 2, wherein further comprising determining a risk associated with heart failure based on the heart rate burden (ΔHR).

5. The method according to claim 2, wherein the endo-phenotype is heart rate burden (ΔHR) and wherein the heat rate burden for a respiratory event is determined as the difference between a maximum pulse rate after an airway opening and a minimum pulse rate during the respiratory event.

6. The method according to claim 5, wherein the respiratory event is one of an apnea or a hypopnea.

7. The method according to claim 1, wherein the pharyngeal collapsibility is determined using a slope of desaturation of the SpO₂ signals.

8. The method according to claim 1, wherein the ventilatory instability is determined based on a slope of an oxygen recovery of the SpO₂ signals.

9. The method according to claim 1, further comprising displaying, using a display, the at least one endo-phenotype for OSA.

10. A method for determining a site of airway collapse for obstructive sleep apnea (OSA) in a subject, the method comprising:
acquiring snoring signals from a subject over a period of time using an acoustic sensor, the snoring signals associated with a plurality of snores;
generating, using a processor, a power spectral density (PSD) plot for each snore in the plurality of snores;
determining, using the processor, at least one snoring feature for each snore in the plurality of snores based on the snoring signals and the PSD plot;
providing the at least one snoring feature for each snore to a machine learning model; and
determining a site of airway collapse for each snore in the plurality of snores using the machine learning model.

11. The method according to claim 10, wherein the site of airway collapse is one of a velum, oropharyngeal lateral walls (OPLW), a tongue base, or an epiglottis,

12. The method according to claim 10, wherein the at least one snoring feature is one or more of relative power in very low (0-125 Hz), low (250-500 Hz) and high (2-2.5 kHz) frequency bands, formant frequencies, harmonic power, and fundamental frequency.

13. The method according to claim 10, wherein the machine learning model is a random forest machine learning model.

14. The method according to claim 10, further comprising determining a treatment for the subject based on the site of airway collapse for at least one snore in the plurality of snores.

15. The method according to claim 10, wherein the period of time corresponds to a sleep time of the subject.

16. The method according to claim 10, further comprising displaying, using a display, the site of airway collapse for each snore in the plurality of snores.

17. The method according to claim 10, wherein the acoustic sensor is a microphone positioned in proximity to the subject.
